(19) United States
(12) Patent Application Publication
(10) Pub. No.: US 2024/0182837 A1
(43) Pub. Date: Jun. 6, 2024

(54) LIPOASPIRATE WASH SYSTEM AND METHOD

(71) Applicant: Sientra, Inc., Santa Barbara, CA (US)

(72) Inventors: Denise Dajles, Santa Barbara, CA (US); Ariel Siedner, Santa Barbara, CA (US); Tom Butz, Santa Barbara, CA (US); Josh Greene, Santa Barbara, CA (US)

(21) Appl. No.: 18/525,191

(22) Filed: Nov. 30, 2023

Related U.S. Application Data
(60) Provisional application No. 63/429,426, filed on Dec. 1, 2022.
(60) Provisional application No. 63/448,895, filed on Feb. 28, 2023.

(51) Int. Cl. C12M 1/26

(52) U.S. Cl. CPC C12M 33/14; C12M 33/22

(57) ABSTRACT

The present disclosure relates to a system for washing lipoaspirate, comprising a chamber; a lid connecting to the top side of the chamber and comprising a harvest port configured to connect to and receive a harvested lipoaspirate from a hose; a sub-assembly comprising a mesh basket and disposed in an upper portion of the chamber, the sub-assembly configured to drain the lipoaspirate through the mesh basket; a tray located in a lower portion of the chamber and comprising a foam assembly configured to concentrate a drained lipoaspirate; and an extraction port along a side wall of the chamber and configured to permit extraction of a concentrated lipoaspirate from the system.

### CROSS-REFERENCE SECTION

This application claims priority to U.S. Provisional Application No. 63/429,426, filed on Dec. 1, 2022, and U.S. Provisional Application No. 63/448,895, filed on Feb. 28, 2023, which are incorporated by reference herein in their entirety for all purposes.

### FIELD OF THE DISCLOSURE

The present disclosure relates, according to some embodiments, to a system for washing lipoaspirate for use in fat grafting procedures along with methods of using said system.

### BACKGROUND

Fat grafting is a common procedure in which adipose tissue, otherwise known as body fat, is removed from one area of the body via liposuction, processed and then transferred to another body area. This technique is used in a variety of reconstructive and aesthetic cases. Some areas where fat grafting is typically used include, face, breast, hips, buttocks, hands and feet but more areas for different applications are currently being explored.

When adipose tissue is harvested, it often includes other agents, such as water or other liquids, free lipids, blood cells, etc., in addition to the adipose cells (adipocytes). It has been found that removal of such impurities and/or fluid from the harvested adipose tissue prior to reintroducing the adipocytes into a patient's body may be beneficial to graft success.

Fat grafting procedures generally include three steps: (1) extraction of the fat from a donor area(s) of a patient, (2) processing of the fat, which involves removing excess tumescent fluid, oils, and blood, thus leaving isolated adipose tissue, and (3) reinjecting the processed fat into the area of interest in the patient's body. Typically devices have used decantation, centrifugation and wash steps during the processing of the fat (step 2) stage to purify the lipoaspirate and have a resulting high percentage of viable adipose tissue for grafting purposes. Several procedures for processing of the fat have been developed, such as that described in U.S. Pat. No. 8,512,695, which is incorporated herein by reference. The '695 patent describes a process for adding a poloxamer 188 (P188) to harvested fat to improve cell viability.

Several procedures and systems have been developed to achieve this separation or “purification” of the fat. For example, U.S. Pat. No. 10,118,140, which is incorporated herein by reference, describes a processing system that includes two chambers—one for collection of lipoaspirate and another for processing of the lipoaspirate. See FIG. 8A of the '140 Patent, which depicts this arrangement.

The use of separate chambers, such as those found in known lipoaspirate washing systems, may be disfavored by some surgeons due to usability issues and a desire to have a closed or semi-closed system where possible. Improvements to the existing multi-chamber systems are desired. Improved methods of manufacturability are also desired.

### SUMMARY

The present disclosure describes a system for washing lipoaspirate that includes a chamber comprising a hollow body formed of side walls having an exterior surface and an interior surface, with the chamber being open at a top side and a bottom side of the chamber. The system may include a lid containing a top surface; a lip extending downward from a bottom surface of the lid and configured to securely connect the lid to the top side of the chamber, and a harvest port including a cylindrical tap extending outwardly from the top side of the lid and configured to connect to and receive a harvested lipoaspirate from a hose. The system may include a sub-assembly comprising a mesh basket and disposed in an upper portion of the chamber, the sub-assembly configured to receive the lipoaspirate received through the harvest port and to separate extracted waste fluid from the lipoaspirate to form a washed lipoaspirate. The system may include a tray configured to insert into a lower portion of the chamber through an opening in the side walls, thereby forming a bottom portion of the system. The tray may include a plate; raised sidewalls positioned along the entire perimeter of the plate and extending upward from a top surface of the plate; and a foam assembly fitting onto the plate and within the sidewalls so that the foam assembly is disposed in a lower portion of the chamber and configured to concentrate the washed lipoaspirate. The system may include an extraction port along one of the side walls of the chamber configured to permit removal of lipoaspirate that has been one of washed and concentrated.

In some embodiments, the extraction port may include a cylindrical hole in the lower portion of the chamber that protrudes outward from the side of the chamber. The extraction port may include threads configured to couple to an extraction port cap that contains the extraction within the system. The lid may include a partition for storing at least one of a spatula and a cleaning packet. The system may include a removable separator positioned on a top surface of the raised sidewalls of the tray, the removable separator configured to separate contents contained within in the chamber from the tray. In some embodiments, the separator may include a gasket configured to form a seal between the removable separator and the chamber; and a handle protruding outward from a side of the removable separator and thus outward from the chamber, wherein the handle is configured to be pulled to remove the removable separator from the chamber. The foam assembly may include a plurality of foam members, forming a complex channel system configured to redistribute a fluid from the lipoaspirate throughout the foam assembly. The foam assembly may be configured to prevent saturation of the foam members before they are able to concentrate the lipoaspirate.

In some embodiments, the system may include a lid. The lid may include an opening on a top surface of the lid configured to permit access to an inside of the mesh basket. The mesh basket may include a mesh bottom forming a bottom portion of the sub-assembly and a plurality of mesh sides forming side portions of the sub-assembly. The tray may include a squeegee. The lid may include a port cap fitting into an opening on a top surface of the lid and may be configured to be removed from the opening permit access to an inside of the chamber by the user with a spatula. The sub-assembly may include a circular hole in a lower portion of the sub-assembly that lines up with extraction port of the chamber. The circular hole may be configured to form a seal with the extraction port through a slit valve.

According to some embodiments, the present disclosure relates to a method for washing a harvested lipoaspirate with a lipoaspirate washing system. The method may include receiving the harvested lipoaspirate into a chamber of the lipoaspirate washing system so that the harvested lipoaspirate is contained by a mesh basket disposed in an upper portion of the chamber of the lipoaspirate washing system to form a received lipoaspirate. The method may include adding a washing solution to the chamber and mixing the washing solution with the received lipoaspirate using a spatula to form a mixed lipoaspirate. The method may include separating the mixed lipoaspirate into an extracted waste fluid and a washed lipoaspirate.

The method may include removing a removable separator from the lipoaspirate washing system so that washed lipoaspirate may contact a foam assembly contained in a tray contained in a lower portion of the chamber and located below the mesh basket. The method may include absorbing a portion of the extracted waste fluid into the foam assembly to concentrate the washed aspirate, forming a concentrated lipoaspirate. In some embodiment, one of the washed lipoaspirate and the concentrated lipoaspirate is removed from the lipoaspirate washing system from an extraction port disposed on a lower portion of a side wall of the chamber. The concentrated lipoaspirate may have a concentration of lipoaspirate per unit volume that is from about 2 to about 100 times higher than the harvested lipoaspirate.

The method may include attaching the harvest port to a liposuction cannula. In some embodiments, adding the washing solution to the chamber may include pouring the washing solution through an opening in a lid that securely connects to a top side of the chamber through a lip extending downward from a bottom surface of the lid. Adding a washing solution to the chamber may be done in a ratio of the washing solution to the received lipoaspirate that ranges from about 1:4 to about 4:1. The mixed lipoaspirate may sit for a time ranging from about 30 seconds to about five minutes. Mixing the washing solution with the received lipoaspirate with a spatula may occur for a time ranging from about 1 minutes to about 10 minutes. The washing solution comprises a biocompatible block copolymer comprising repeating units of Polyethylene oxide (PEO) and polypropylene oxide (PPO).

The method may include receiving additional harvested lipoaspirate into the chamber of the lipoaspirate washing system so that the additional harvested lipoaspirate is contained by the mesh basket disposed in the upper portion of the chamber of the lipoaspirate washing system to form additional received lipoaspirate. The method may include adding additional washing solution to the chamber and mixing the washing solution with the received lipoaspirate with the spatula to form additional mixed lipoaspirate. The method may include separating additional waste fluid from the additional mixed lipoaspirate to form additional extracted waste fluid and additional washed lipoaspirate, wherein the extracted waste fluid is removed from the lipoaspirate washing system from an extraction port disposed on a lower portion of a side wall of the chamber.

### BRIEF DESCRIPTION OF THE DRAWINGS

Some embodiments of the disclosure may be understood by referring, in part, to the present disclosure and the accompanying drawings, wherein:

FIG. 1 illustrates an exploded view of a disclosed lipoaspirate wash system, according to an example embodiment of the disclosure;

FIG. 2A illustrates a top view of the lipoaspirate wash system, according to an example embodiment of the disclosure;

FIG. 2B illustrates an isometric view of the lipoaspirate wash system of FIG. 2A, according to an example embodiment of the disclosure;

FIG. 3A illustrates a top view of the lipoaspirate wash system of FIG. 2A that is connected to a harvesting hose, according to an example embodiment of the disclosure;

FIG. 3B illustrates a top view of the lipoaspirate wash system of FIG. 2A having the port cap removed, according to an example embodiment of the disclosure;

FIG. 3C illustrates a top view of the lipoaspirate wash system of FIG. 2A having the lid removed, according to an example embodiment of the disclosure;

FIG. 4A illustrates a top view of a foam configuration with a channel running along the perimeter, according to an example embodiment of the disclosure;

FIG. 4B illustrates a top view of the foam tray of FIG. 4A with a channel system and foam, according to an example embodiment of the disclosure;

FIG. 5 illustrates a top view of a foam assembly having channels, according to an example embodiment of the disclosure;

FIG. 6A illustrates a side view of a removable separator, according to an example embodiment of the disclosure;

FIG. 6B illustrates a top view of the removable separator of FIG. 6A, according to an example embodiment of the disclosure;

FIG. 6C illustrates an isometric view of the removable separator of FIG. 6A, according to an example embodiment of the disclosure;

FIG. 7A illustrates an isometric view of a squeegee, according to an example embodiment of the disclosure;

FIG. 7B illustrates a side view of the squeegee from FIG. 7A, according to an example embodiment of the disclosure;

FIG. 7C illustrates another side view of the squeegee from FIG. 7A, according to an example embodiment of the disclosure;

FIG. 7D illustrates a top view of the squeegee from FIG. 7A:

FIG. 8 illustrates a top view of a sub-assembly, according to an example embodiment of the disclosure;

FIG. 9A illustrates a side view of a chamber, according to an example embodiment of the disclosure;

FIG. 9B illustrates a top view of the chamber from FIG. 9A, according to an example embodiment of the disclosure;

FIG. 10 illustrates a side view of the lipoaspirate washing system of FIG. 2A having a slightly tiled chamber, according to an example embodiment of the disclosure;

FIG. 11 illustrates an envelope storage system for a disclosed lipoaspirate washing system, according to an example embodiment of the disclosure;

FIG. 12 illustrates a box storage system for a disclosed lipoaspirate washing system, according to an example embodiment of the disclosure;

FIG. 13A illustrates a front view of the lipoaspirate washing system of FIG. 2A, according to an example embodiment of the disclosure;

FIG. 13B illustrates a top view of the lipoaspirate washing system of FIG. 2A, according to an example embodiment of the disclosure;

FIG. 13C illustrates a side view of the lipoaspirate washing system of FIG. 2A, according to an example embodiment of the disclosure;

FIG. 14A illustrates an isometric view of a sub-assembly and squeegee fitting into a chamber, according to an example embodiment of the disclosure;

FIG. 14B illustrates another isometric view of the chamber of FIG. 14A, according to an example embodiment of the disclosure;

FIG. 14C illustrates a sectional view of the chamber of FIG. 14A, according to an example embodiment of the disclosure;

FIG. 15A illustrates a top view of a foam assembly, according to an example embodiment of the disclosure;

FIG. 15B illustrates an exploded view of the foam assembly of FIG. 15A, according to an example embodiment of the disclosure;

FIG. 15C illustrates a side view of the foam assembly of FIG. 15A, according to an example embodiment of the disclosure;

FIG. 16A illustrates an isometric view of a port cap, according to an example embodiment of the disclosure;

FIG. 16B illustrates a top view of the port cap of FIG. 16A, according to an example embodiment of the disclosure;

FIG. 16C illustrates a side sectional view of the port cap of FIG. 16A, according to an example embodiment of the disclosure;

FIG. 16D illustrates a side view of the port cap of FIG. 16A, according to an example embodiment of the disclosure;

FIG. 16E illustrates a front sectional view of the port cap of FIG. 16A, according to an example embodiment of the disclosure;

FIG. 16F illustrates a side sectional view of the port cap of FIG. 16A, according to an example embodiment of the disclosure;

FIG. 17A illustrates a top view of a lid, according to an example embodiment of the disclosure;

FIG. 17B illustrates a top isometric view of the lid of FIG. 17A, according to an example embodiment of the disclosure;

FIG. 17C illustrates a side sectional view of the lid of FIG. 17A, according to an example embodiment of the disclosure;

FIG. 17D illustrates a side view of the lid of FIG. 17A, according to an example embodiment of the disclosure;

FIG. 17E illustrates a bottom view of the lid of FIG. 17A, according to an example embodiment of the disclosure;

FIG. 17F illustrates a bottom isometric view of the lid of FIG. 17A, according to an example embodiment of the disclosure;

FIG. 18A illustrates a top view of a lid, according to an example embodiment of the disclosure;

FIG. 18B illustrates a top isometric view of the lid of FIG. 18A, according to an example embodiment of the disclosure;

FIG. 18C illustrates a side view of the lid of FIG. 18A, according to an example embodiment of the disclosure;

FIG. 19A illustrates an isometric view a chamber, according to an example embodiment of the disclosure;

FIG. 19B illustrates a top view of the chamber of FIG. 19A, according to an example embodiment of the disclosure;

FIG. 19C illustrates a side sectional view of the chamber of FIG. 19A, according to an example embodiment of the disclosure;

FIG. 19D illustrates a front view of the chamber of FIG. 19A, according to an example embodiment of the disclosure;

FIG. 19E illustrates a side view of the chamber of FIG. 19A, according to an example embodiment of the disclosure;

FIG. 19F illustrates a sectional view of an end of the chamber of FIG. 19A, according to an example embodiment of the disclosure;

FIG. 19G illustrates a bottom view of the chamber of FIG. 19A, according to an example embodiment of the disclosure;

FIG. 19H illustrates a side sectional view of the chamber of FIG. 19A, according to an example embodiment of the disclosure;

FIG. 20A illustrates a front sectional view of a chamber, according to an example embodiment of the disclosure;

FIG. 20B illustrates a top view of the chamber of FIG. 20A, according to an example embodiment of the disclosure;

FIG. 20C illustrates a rear sectional view of the chamber of FIG. 20A, according to an example embodiment of the disclosure;

FIG. 20D illustrates a front view of the chamber of FIG. 20A having the tray installed, according to an example embodiment of the disclosure;

FIG. 20E illustrates a front view of the chamber of the chamber of FIG. 20A without the tray installed, according to an example embodiment of the disclosure;

FIG. 21A illustrates a side view of a chamber, according to an example embodiment of the disclosure;

FIG. 21B illustrates a front view of the chamber of FIG. 21A having a tray installed, according to an example embodiment of the disclosure;

FIG. 21C illustrates an alternative side view of the chamber of FIG. 21A having a tray installed, according to an example embodiment of the disclosure;

FIG. 21D illustrates a back view of the chamber of FIG. 21A, according to an example embodiment of the disclosure;

FIG. 22A illustrates an isometric view of a sub-assembly, according to an example embodiment of the disclosure;

FIG. 22B illustrates a top view of the sub-assembly of FIG. 22A, according to an example embodiment of the disclosure;

FIG. 22C illustrates a front view of the sub-assembly of FIG. 22A, according to an example embodiment of the disclosure;

FIG. 22D illustrates a side view of the sub-assembly of FIG. 22A, according to an example embodiment of the disclosure;

FIG. 22E illustrates a back view of the sub-assembly of FIG. 22A, according to an example embodiment of the disclosure;

FIG. 22F illustrates an alternative side view of the sub-assembly of FIG. 22A, according to an example embodiment of the disclosure;

FIG. 23A illustrates an isometric view of a tray, according to an example embodiment of the disclosure;

FIG. 23B illustrates a top view of the tray of FIG. 23A, according to an example embodiment of the disclosure;

FIG. 23C illustrates a side view of the tray of FIG. 23A, according to an example embodiment of the disclosure;

FIG. 23D illustrates a top isometric view of the tray of FIG. 23A, according to an example embodiment of the disclosure;

FIG. 23E illustrates a front view of the tray of FIG. 23A, according to an example embodiment of the disclosure;

FIG. 23F illustrates a front sectional view of the tray of FIG. 23A, according to an example embodiment of the disclosure;

FIG. 23G illustrates a bottom view of the tray of FIG. 23A, according to an example embodiment of the disclosure;

FIG. 23H illustrates a zoomed in view of part of the tray of FIG. 23A, according to an example embodiment of the disclosure;

FIG. 23I illustrates a front sectional view of the tray of FIG. 23A, according to an example embodiment of the disclosure;

FIG. 23J illustrates a zoomed in view of a corner of the front sectional view shown in FIG. 23I, according to an example embodiment of the disclosure;

FIG. 24A illustrates a side view of a removable separator, according to an example embodiment of the disclosure;

FIG. 24B illustrates an alternate side view of the removable separator of FIG. 24A, according to an example embodiment of the disclosure;

FIG. 24C illustrates an isometric view of the removable separator of FIG. 24A, according to an example embodiment of the disclosure;

FIG. 24D illustrates a top view of the removable separator of FIG. 24A, according to an example embodiment of the disclosure;

FIG. 24E illustrates a bottom isometric view of the removable separator of FIG. 24A, according to an example embodiment of the disclosure;

FIG. 24F illustrates a bottom view of the removable separator of FIG. 24A, according to an example embodiment of the disclosure; and

FIG. 24G illustrates a front view of the removable separator of FIG. 24A, according to an example embodiment of the disclosure.

### DETAILED DESCRIPTION

The present disclosure relates to lipoaspirate wash systems and methods for washing harvested lipoaspirate using the wash system. Disclosed lipoaspirate wash systems advantageously include a single chamber construction permitting both the collection and processing of lipoaspirate. Disclosed lipoaspirate wash systems may be used in an operating room to aspirate, harvest, wash, filter, and transfer adipose tissue for reinjection into the same patient that the lipoaspirate was harvested from, with no additional processing needed. Disclosed systems and methods may be used in various surgical specialties when aspiration of adipose tissue is desired, including plastic and reconstructive surgery, neurosurgery, gastrointestinal and affiliated organ surgery, urological surgery, general surgery, orthopedic surgery, gynecological surgery, thoracic surgery, and laparoscopic surgery.

### Lipoaspirate Washing Systems

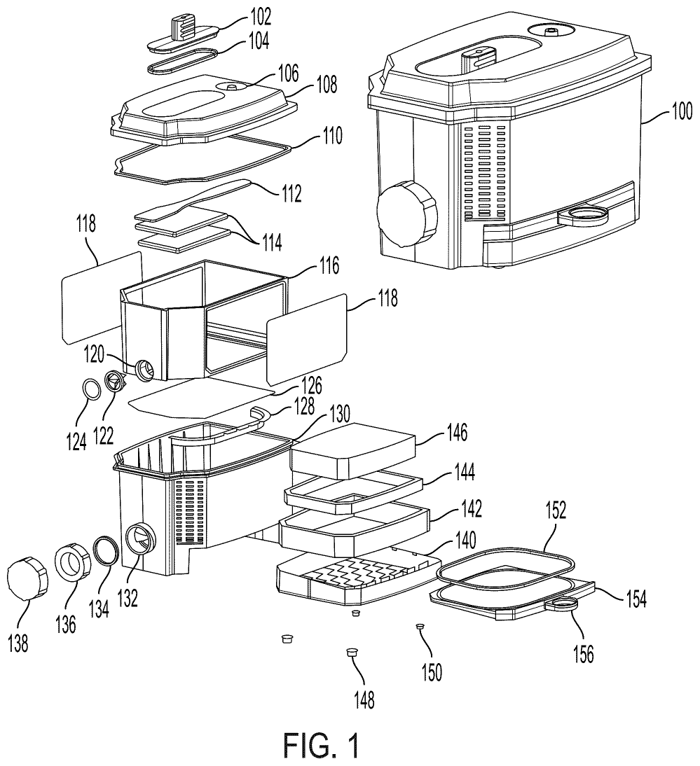

FIG. 1 illustrates an exploded view of a disclosed lipoaspirate washing system 100. As shown in FIG. 1, the system 100 may include a chamber 130 having a hollow body formed of side walls having an exterior surface and an interior surface, the chamber being open at a top side and a bottom side of the chamber. The opening at the top side of the chamber 130 may be used to add lipoaspirate to the system 100 so that it can be washed. Disclosed systems 130 advantageously use a single chamber 130 instead of multiple chambers used by known systems. In some embodiments, chamber 130 may be graduated so that a user may determine the volume of materials inside of the chamber 130, as shown in FIG. 1. The chamber 130 may be made of any material, including polymers (e.g., high density polyethylene, polypropylene), metals, and combinations thereof. The chamber 130 may be configured to contain or support the remaining components of the system 100, such as lids 108 and trays 140.

So that washed and/or concentrated lipoaspirate may be removed from a system 100, a chamber 130 may include an extraction port 132. The washed lipoaspirate and the concentrated lipoaspirate may be extracted from the system 100 through the extraction port 132. The extraction port 132 may include a cylindrical hole in a lower portion of the chamber 130 that protrudes outward from the side of the chamber 130. The extraction port 132 may couple to a hose, syringe, or other extraction device to facilitate extraction of the washed lipoaspirate and the concentrated lipoaspirate from the system 100. The extraction port 132 may contain threads configured to couple to an extraction port cap 136 as well as an extraction port cosmetic cap 138, which plug the extraction port 132 when not being used to remove washed lipoaspirate from the system 100. The extraction port 132 may include an extraction port gasket 134 providing a seal between the extraction port 132 and the extraction port cap 136 and/or extraction port cosmetic cap 138. The seal provided by the extraction port gasket 134 may prevent accidental leakage from the system 100. In some embodiments, the chamber 130 may include a plurality of extraction ports 132. Not pictured specifically in FIG. 1 is a waste drain port that may be used to extract waste fluids from the system.

A disclosed system 100 has various configurations, such as having an open, closed, or semi-closed configuration. An open configuration is one where a lid 108 is not attached to the chamber 130 of the system 100. The lid 108 may couple to a top of the chamber 130, thereby providing for the closed or semi-closed top cover for the system 100. The lid 108 may include a top surface and a bottom surface. The lid 108 may include a lip extending downward from a bottom surface of the lid and be configured to securely connect the lid to the top side of the chamber 130. On a top surface of the lid 108, the lid 108 may include a harvest port 106. The harvest port 106 may include a cylindrical tap extending outwardly from the top side of the lid 108 and be configured to connect to and receive a harvested lipoaspirate from a hose or other source. Harvested lipoaspirate includes any lipoaspirate derived from a human or animal source, such as through liposuction. Harvesting of lipoaspirate may be achieved through known methods, such as by liposuction machines or manual harvesting. The harvest port 106 may be threaded or smooth as to couple with various types of hose or direct-to-device couplings. The harvest port 106 may recess, be flush with, or protrude from a top surface of the lid 108. FIG. 1 shows a recessed harvest port 106 configuration. In some embodiments, the lid 108 may include a plurality of harvest ports 106, such as having from 1-5 harvest ports 106, or more.

As shown in FIG. 1, a disclosed system 100 may include a lid 108 having an opening that may provide a user with access to contents of the system 100. For example, the lid 108 opening may permit a user to add fluids (e.g., washing fluids) and harvested lipoaspirate to the system 100. During a washing process, a user may access the contents of the system 100 through the lid 108, such as by doing so with a spatula 112 for mixing contents. The lid 108 opening may advantageously provide for the system 100 being semi-closed or closed, depending on user preference.

In its semi-closed configuration, the system 100 incorporates a lid 108 having a small removable port cap 102. The port cap 102 can advantageously be used to add fluids, and other substances such as an admixture of a cleaning packet and saline, to the lipoaspirate and to use a tool, such as a spatula 112, to mix and wash the harvested lipoaspirate with the fluids, or P188 and saline admixture. The lid 108 may include a port gasket 104 that provides a seal in between the port cap 102 and the lid 108. In some embodiments, the lid 108 may be configured to have more than one opening and therefore more than one port cap 102. For example, the lid 108 may be configured to have one or more port caps 102, such as from 1-5 port caps 102, or more. In a closed configuration, system 100 may have a lid without the port cap 102 or run without removing the port cap 102.

As shown in FIG. 1, the system 100 may include a spatula 112 and one or more cleaning packets 114. Each cleaning packet may be mixed with a fluid (e.g., saline) to prepare a washing solution for washing a harvested lipoaspirate. The cleaning packets 114 may include any surfactants known in the art for washing a lipoaspirate. For example, the cleaning packets 114 may include a biocompatible block copolymer, such as a poloxamer surfactant having repeating units of polyethylene oxide (PEO) and polypropylene oxide (PPO). The disclosed system 100 may include cleaning packets 114 containing compositions as disclosed in U.S. Pat. No. 8,512,695. The polymer surfactant preferably includes P188. The cleaning packets 114 may include a saline component, including any known salt, such as sodium chloride, potassium chloride, and other salts. The spatula 112 may be made of any known materials including polymers (e.g., polyethylene, polypropylene) and metal.

As shown in FIG. 1, the system 100 for washing a lipoaspirate may include a sub-assembly 116 including a mesh basket having an opening at the top. The sub-assembly 116 may be configured to seat into an upper portion of a chamber 130. The sub-assembly, in some embodiments, may receive a harvested lipoaspirate through the opening at the top and contain the harvested lipoaspirate with its mesh basket during any washing or processing steps. The mesh basket permits waste fluids to separate away from the lipoaspirate as it gets washed. The sub-assembly 116 may receive the harvested lipoaspirate through the harvest port 106 of the lid 108. The mesh basket of the sub-assembly 116 may include a mesh bottom 126 forming a bottom portion of the sub-assembly. The mesh basket may have a plurality of mesh sides 118 that form side portions of the sub-assembly. The mesh size of the mesh basket may include a mesh ranging from about 2 mesh (e.g., 11,100 micron) to about 400 mesh (e.g., 37 micron). For example, the mesh size may be about 2 mesh, or about 2 mesh, or about 50 mesh, or about 100 mesh, or about 150 mesh, or about 200 mesh, or about 250 mesh, or about 300 mesh, or about 350 mesh, or about 400 mesh, where about includes plus or minus 25 mesh.

According to some embodiments, as shown in FIG. 1, a disclosed sub-assembly may include a circular hole 120 at a lower portion of the sub-assembly. As shown in FIG. 1, the circular hole 120 may seat in line with an extraction port 132 of the chamber 130. The circular hole 120 may also seat above the extraction port 132. In some embodiments, the circular hole 120 may be fitted with a slit valve 122 and a slit lock 124, which may form a seal or partial seal with the extraction port 132, permitting extraction of washed lipoaspirate and concentrated lipoaspirate from the sub-assembly to outside of the chamber 130 through extraction port 132.

As shown in FIG. 1, the disclosed lipoaspirate washing system 100 may include a tray 140 configured to insert into a lower portion of the chamber 130 through an opening in the side walls, thereby forming a bottom portion of the system 100. As a lipoaspirate is washed, washings fall away from the lipoaspirate contained in the sub-assembly and down into the tray 140. The tray 140 may include a plate that is generally flat in shape and having raised sidewalls positioned along the entire perimeter of the plate and extending upward from a top surface of the plate. A top surface of the plate may have various textures, such as those shown in shown in FIG. 1. The various texture may promote fluid drainage. The tray 140 may include a foam 146 held into place by container elements 142 and 144. The foam 146 may seat within the sidewalls of the tray 140 and within the container elements 142 and 144. In some embodiments, the foam 146 may have a thickness ranging from about 0.5 inches to about 3 inches. The foam 146 may have a thickness of about 0.5 inches, or about 1 inch, or about 1.5 inches, or about 2 inches, or about 2.5 inches, or about 3 inches, where about includes plus or minus 0.25 inches. The foam 146 may have an absorptive capacity ranging from about 25 g/100 cm² (at 3 mm thickness) to about 75 g/100 cm². The foam 146 may have an absorptive capacity of about 25 g/100 cm², or about 30 g/100 cm², or about 35 g/100 cm², or about 40 g/100 cm², 45 g/100 cm², or about 50 g/100 cm², or about 55 g/100 cm², or about 60 g/100 cm², or about 65 g/100 cm², or about 70 g/100 cm², or about 75 g/100 cm², at a foam thickness of 3 mm, where about includes plus or minus 2.5 g/100 cm². In some embodiments, the foam 146 may have an absorptive capacity of about 47.9 g/100 cm² at a thickness of 3 mm. The foam 146 may have an area ranging from about 100 cm² to about 500 cm². For example, the foam 146 may have an area of about 100 cm², or about 150 cm², or about 200 cm², or about 250 cm², or about 300 cm², or about 350 cm², or about 400 cm², or about 450 cm², or about 500 cm². In some embodiments, the foam 146 may have an area of 182 cm². When the system 100 is assembled, the foam 146 may be disposed in a lower portion of the chamber 130, right below a sub-assembly 116. During a washing process, the washed lipoaspirate may come into contact with the foam 146, where the washed lipoaspirate is concentrated. Disclosed foam 146 advantageously concentrates the washed lipoaspirate by absorbing such waste and wash fluids.

Disclosed foam 146 may include a plurality of foam members, forming a complex channel system configured to redistribute fluid from the lipoaspirate throughout the foam, thereby concentrating the lipoaspirate. In some embodiments, disclosed foam 146 advantageously prevents saturation of the foam when absorbing fluids from the lipoaspirate, thereby concentrating the lipoaspirate. In some embodiments, the tray 140 may include a plurality of adhesive-back bumpers 148, 150 that prevent sliding of the tray 140 and absorb shock for the tray 140 and the system 100. The bumpers 148, 150 may prevent the system 100 from moving around, therefore keeping the system 100 steady on a surface such as a table or floor. In some embodiments, the bumpers 148, 150 may have varying heights from each other to give the system 100 a slight incline to guide fluid (e.g., waste fluid and washed lipoaspirate) to various parts of the system 100. The adhesive-back bumpers 148, 150 may be of various shapes and sizes, including having an outside diameter of about ⅛ inch, or about ¼ inch, or about ⅜ inch, or about ½ inch, or about ⅝ inch, or about ¾ inch, where about includes plus or minus 1/16 inch. The adhesive-back bumpers 148, 150 may have a height of about ¼ inches, or about ⅜ inch, or about ½ inch, or about ¾ inch, or about 1 inch, where about includes plus or minus ⅛ inch.

A user may want to precisely control when the washed lipoaspirate leaves the sub-assembly 116 (upper portion) of the system 100 to drop down to the tray (lower portion) of the system 100. By removing a removable separator 154 from the system 100, the washed lipoaspirate collects into the tray 140, thereby contacting the foam 146. The removable separator 154 may be positioned on a top surface of the raised sidewalls of the tray 140 and includes a gasket 152 configured to form a seal between the removable separator 154 and either one of the chamber 130 and the tray 140. The removable separator 154 may include a handle 156 protruding outwardly from a side of the removable separator 154 and thus outward from the chamber 130, wherein the handle 156 is configured to be pulled to remove the removable separator 154 from the chamber 130. A system 100 may include a squeegee 128 configured to prevent fluids from leaking from the system 100 while the removable separator 154 is being removed from the system 100. The removable separator 154 may include any number of gaskets to aid in sealing spaces in between the removable separator 154 and other system 100 components while the removable separator 154 is coupled to the system. The gasket may provide air and fluid sealing capabilities.

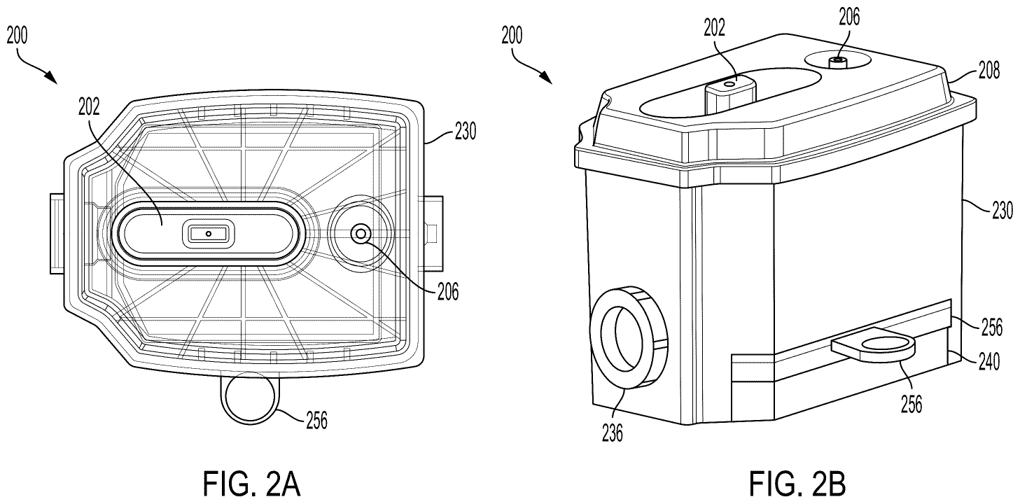

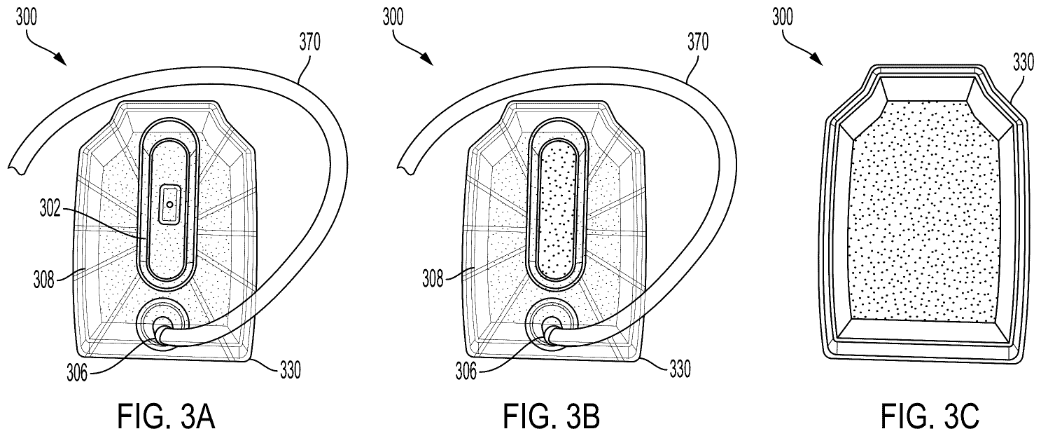

Component numbers increase incrementally based on which figure they are depicting. For example, component 100 in FIG. 1 will be annotated as component 200 in FIG. 2 and as component 300 in FIG. 3. FIGS. 2A and 2B illustrate a top view and an isometric view of a lipoaspirate washing system 200, where the components shown in FIG. 1 have been assembled together. As shown in FIGS. 2A and 2B, a lid 208 may attach to a chamber 230. FIGS. 2A and 2B depict an embodiment where the port cap 202 is inserted into the lid 208. Similarly, as shown in FIGS. 3A-3C, a user may remove one of the port cap 302 or lid 308 to have various types of access to the inside of lipoaspirate washing system 300. Being able to remove either of the port cap 302 or lid 308 gives the user higher flexibility in use of the system (e.g., adding an admixture of saline and P188 to the harvested lipoaspirate, and for mixing the P188 and saline admixture with the harvested fat using mixing device, such as spatula 112) but also reduces the surface area of direct exposure to air. In terms of surface area, the direct overhead exposure to air is reduced (FIG. 3B) relative to prior art systems and can quickly be reduced to 0% by placing the port cap 302 on the opening (FIG. 3A) while the user is not mixing or washing. In the open system modality (FIG. 3C), the user simply removes the lid 308 covering the system 300 and has complete unobstructed access to the lipoaspirate.

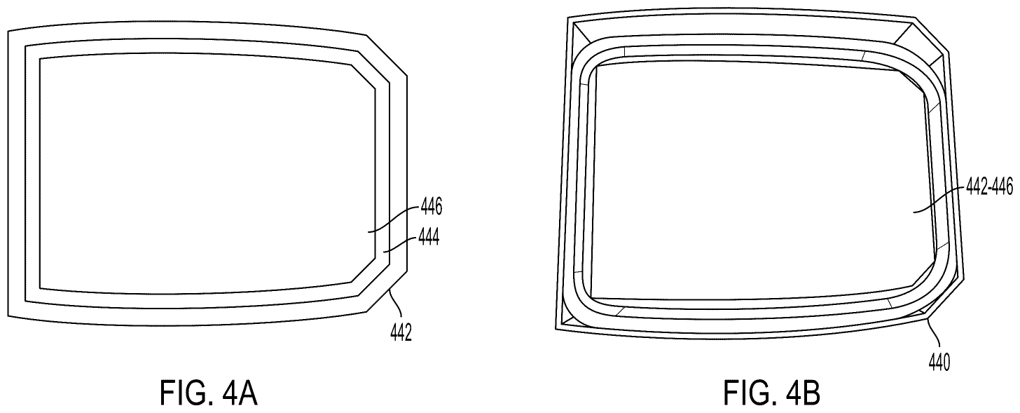

FIGS. 4A and 4B illustrate a foam configuration with a channel along the perimeter. The system 400 may include a highly absorbent foam 446 disposed in tray 440 and held into place by container elements 442 and 444. The foam 446 may be initially separated from the mesh basket of the chamber by a removable separator that is adapted to be removed from the chamber by simply pulling out the removable separator to allow the mesh basket to contact the foam 446. The foam 446 has an advanced foam geometry to maximize fluid absorption and produce highly concentrated fat. After washing the harvested lipoaspirate, the washed lipoaspirate may be concentrated by placing it onto a foam 446 contained in the tray 440. During the concentration step (through use of the foam) in the washing methods, the single chamber device may be able to handle a large amount of fluid and oil that potentially remains after separating the waste fluids from the washed lipoaspirate and at the same time concentrate the remaining lipoaspirate as quickly as possible. In order to control this large volume of oil and fluid, the foam 446 may include the channel running along its entire perimeter that increases the surface area of foam to fluid interphase as well as acting as a reservoir for large sudden discharges at the beginning of the concentration step. The added surface area allows for the foam of the foam 446 to expand quickly and make contact with the overlying mesh. This contact process between foam and mesh ensures the lipoaspirate concentrates, as shown in FIGS. 4A and 4B.

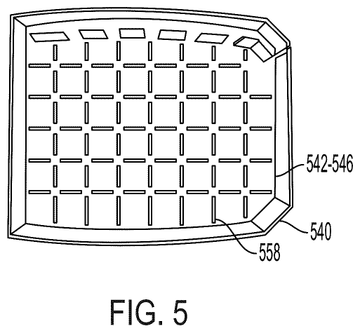

In some embodiments, as shown in FIG. 5, the foam 546, held into place by container members 542 and 544, may include a complex channel system 558 to redistribute fluid throughout the foam 546. This allows for at least two benefits. First, the channel system 558 ensures the foam's 546 absorption capacity is maximized by extending the surface area of foam 546 to fluid interphase. Second, it allows excess fluid to pool in areas of the foam 546 of a tray and prevent saturating the foam before it is able to concentrate the lipoaspirate.

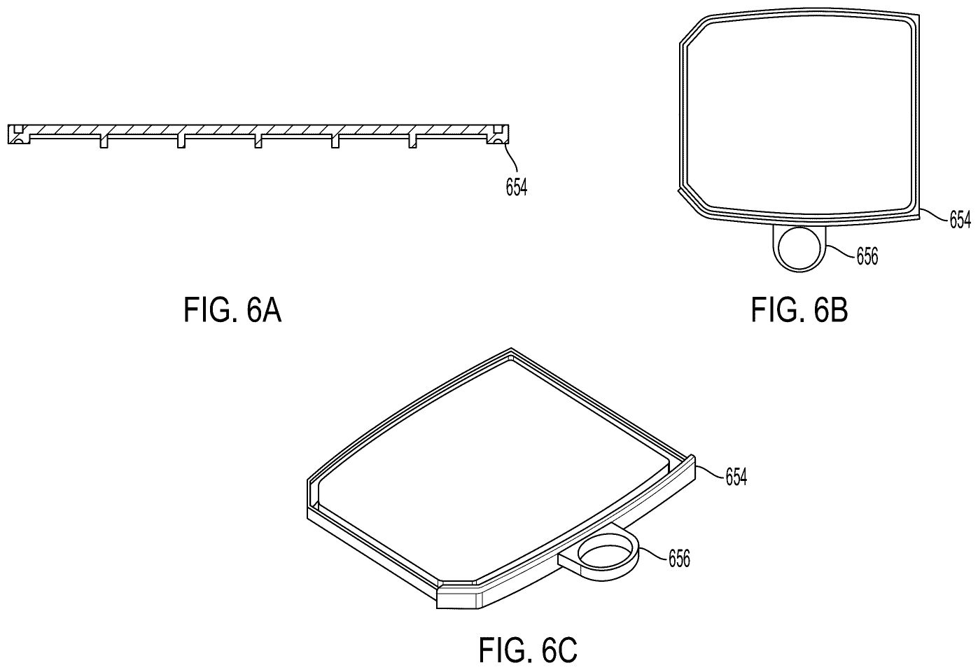

As shown in FIGS. 6A-C, the disclosed system 600 may include a removable separator 654 having a handle 656. After washing of the harvested lipoaspirate, the removable separator 654 may be removed, which allows for the washed lipoaspirate to contact the foam. The foam concentrates the washed lipoaspirate, forming a concentrated lipoaspirate. FIG. 6 illustrates a side, top, and isometric view of the removable separator 654. The removable separator 654 facilitates usage of only a single chamber 130 (as shown in FIG. 1) to accommodate both of a washing step and concentration step in treating the lipoaspirate for reinjection into the patient. The removable separator 654 ensures that the lipoaspirate is properly washed and drained before it is exposed to the concentrating foam. The removable separator 654 also advantageously facilitates both an airtight seal and a fluid seal, and is able to support a vacuum without significant deflection, and doing so in a tightly constrained space and facilitating easy removal by the user.

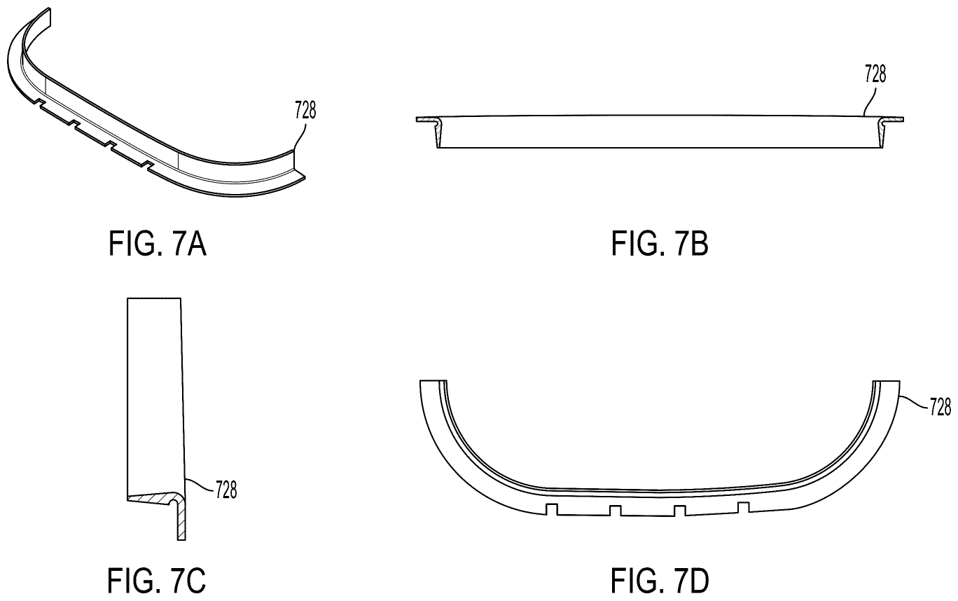

In some embodiments, a system may also include a self-cleaning feature to clean the removable separator as it is removed from the system. As shown in FIG. 7, a system may include a squeegee 728 as a self-cleaning feature. The squeegee 728 may be an L-shaped or U-shaped bracket with one side bent in a quarter circle followed by a straight length and then a second quarter-circle bend. The squeegee 728 is configured to sit into a portion of the chamber so that it is held in place while part of its body hangs just above a top surface of the removable separator and foam. By removing the removable separator from the chamber, the removable separator slides against a bottom surface of the squeegee 728, thereby removing waste from a top surface of both of the removable separator. The squeegee 728 and removable separator prevent lipoaspirate and waste products from leaking out of the system and directs the washed lipoaspirate to the foam. In some embodiments, removal of the removable separator may clean the removable separator via a squeegee-like action from the squeegee 728. The removable separator couple with the squeegee 728 may help prevent system leakage in the event of an overflow.

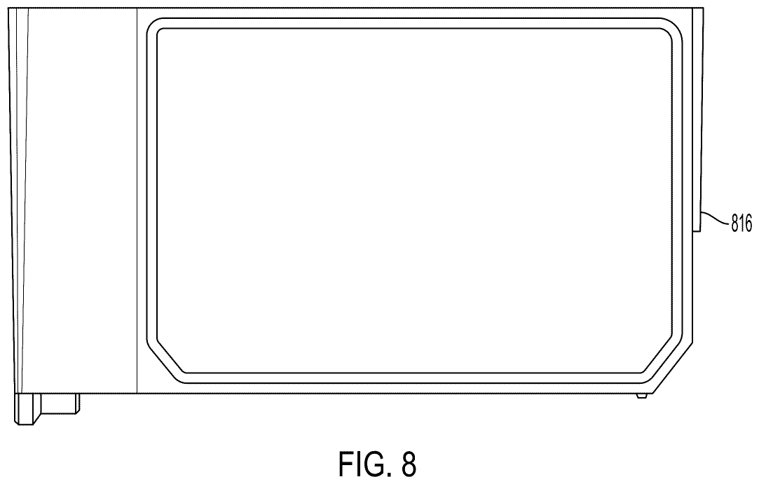

FIG. 8 illustrates a top view of a disclosed sub-assembly 816. A disclosed system may include a fluid draining management system through a variety of components and features of the design of a disclosed sub-assembly 816. As shown in FIG. 8, the sub-assembly 816 may have a 45 degree (other angles are contemplated) cut feature in the back of the sub-assembly. The sub-assembly 816 may also have slight raise in the back of its frame to increase flow to the drain.

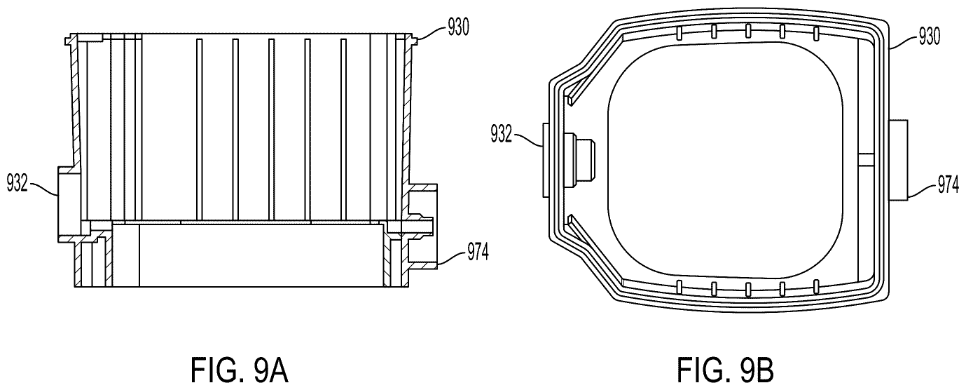

FIGS. 9A and 9B illustrate a side view and top view of a chamber 930. The chamber 930 may include a waste drain port 974 configured to permit removal of waste fluids out of the system as they are separated from lipoaspirate during the washing processes. The waste drain port 974 may be positioned opposite the extraction port 932 and may be positioned lower in height in comparison to the extraction port 932. For example, the waste drain port 974 may be further away from the lid than the extraction port 932. The waste drain port 974 may include any type of known closing mechanism to begin and end draining of the waste fluid from the system. In some embodiments, suction may be applied to a waste drain port 974, such as by a vacuum, which may facilitate removal of waste fluids from the system.

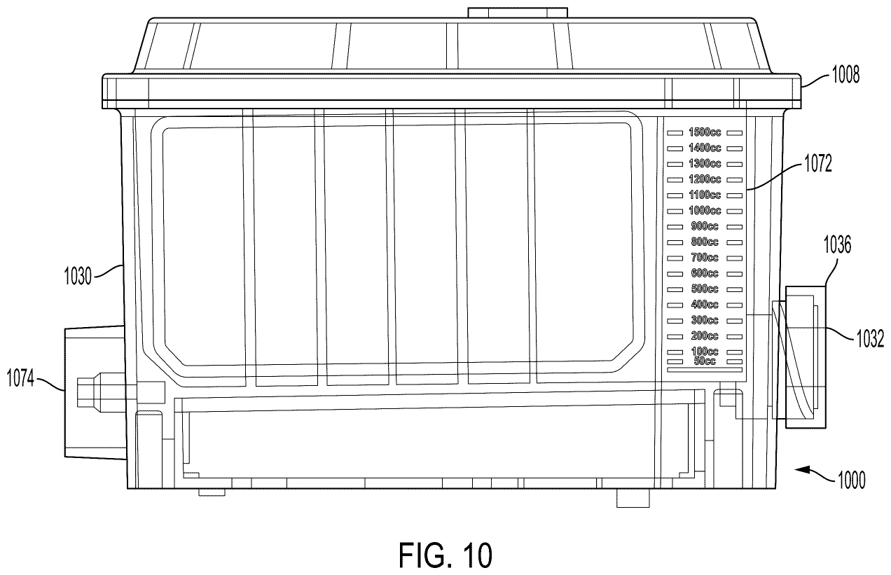

In some embodiments, as shown in FIG. 10, a chamber 1030 of a system 1000 may be slightly tilted towards the waste drain port 1074, which may advantageously permit better collection of waste fluid from the system 1000. FIG. 10 also shows extraction port 1074 on the opposite side of the system 1000 with respect to the waste drain port 1074. As shown in FIG. 10, the waste drain port 1074 may be positioned lower than the extraction port 1032. In some embodiments, the tilt may involve a tilt of the system and/or a tilt of the basket. In some embodiments, the system may be tilted towards the waste drain port 1074. To aid with extraction of the washed lipoaspirate, the basket and/or sub-assembly may be tilted towards the extraction port 1074.

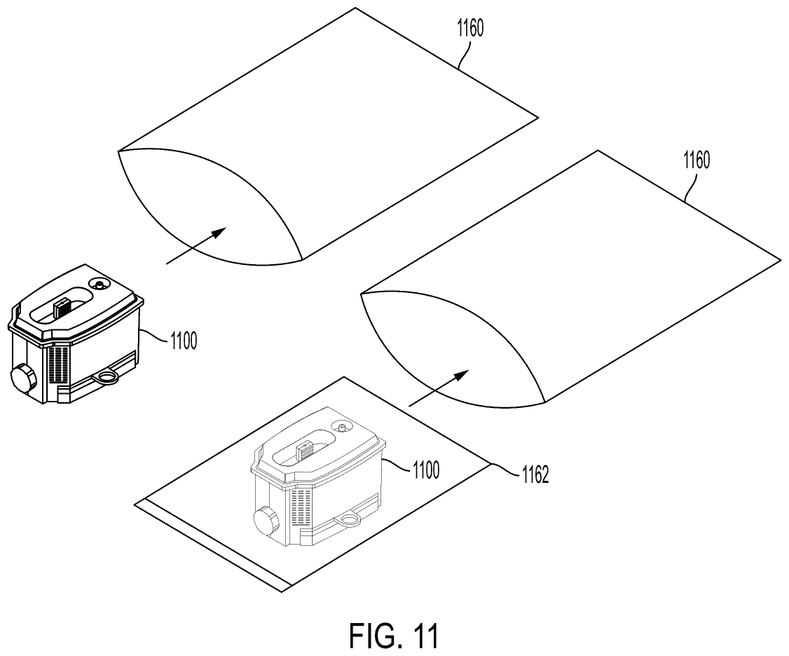

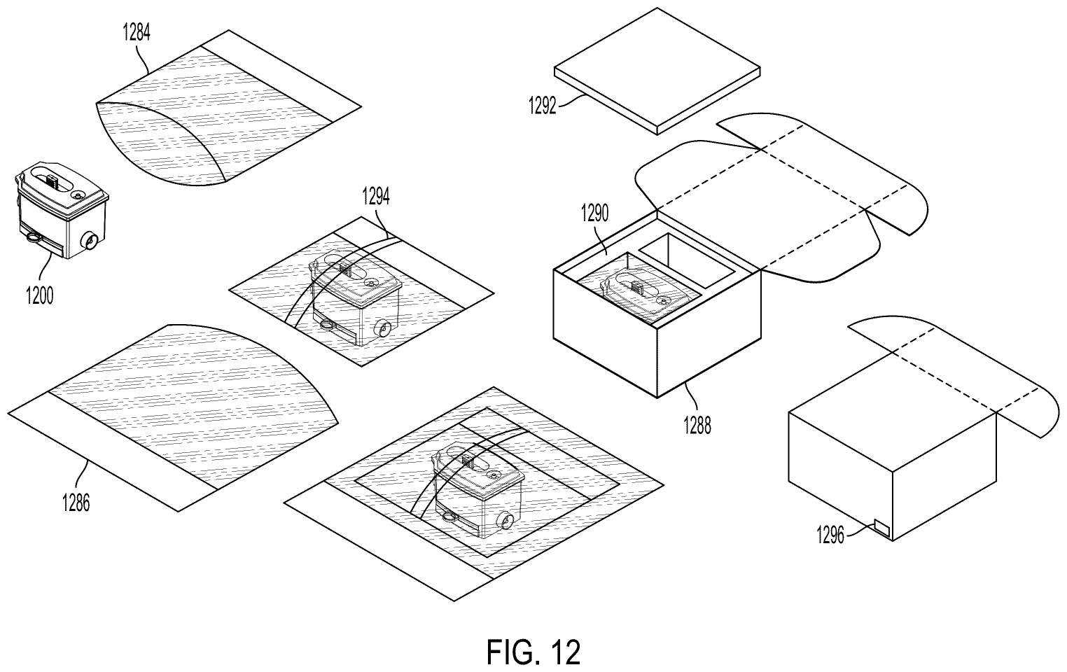

According to some embodiments, a disclosed system 1100 for washing a lipoaspirate may be stored in a container for storage or transport, as shown in FIG. 11. For example, a system 1100 may be placed onto a sheet 1162 and then placed into an envelope 1160. Both the sheet 1162 and envelop 1160 may be made of various materials including paper, plastic, metal, and a mixture thereof. Similarly, as shown in FIG. 12, the system 1200 may be placed in an inner bag 1284. A pouch handle 1294 may attach from one end of the inner bag 1284 to another end of the inner bag 1284, thereby securing the system 1200 inside. The pouch handle 1294 may permit a user to carry the system 1200 contained inside the inner bag 1284 without touching the exterior of the inner bag 1284. The secured system 1200 and inner bag 1284 may together be added to an outer bag 1286. Each of the inner bag 1284 and outer bag 1286 may be sterile. The outer bag 1286 containing the inner bag 1284 and system 1200 may be inserted into a carton 1288 containing a foam insert 1290. Once secured inside the carton 1288, a foam top layer 1292 may be placed on top to provide a top surface protection of the system 1200. A device identification label 1296 may be placed on an outside surface of the carton 1288 for identification and tracking.

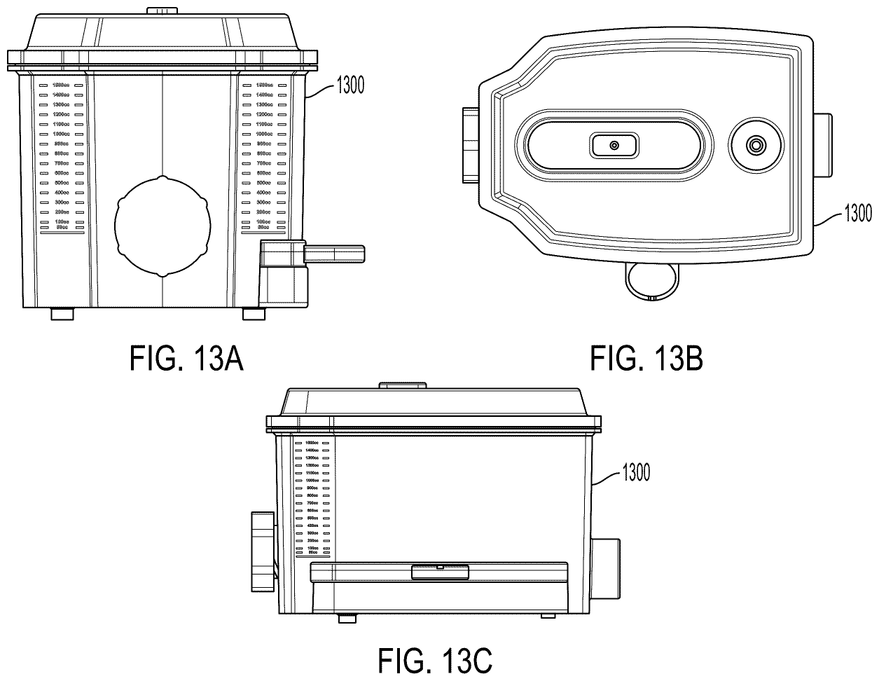

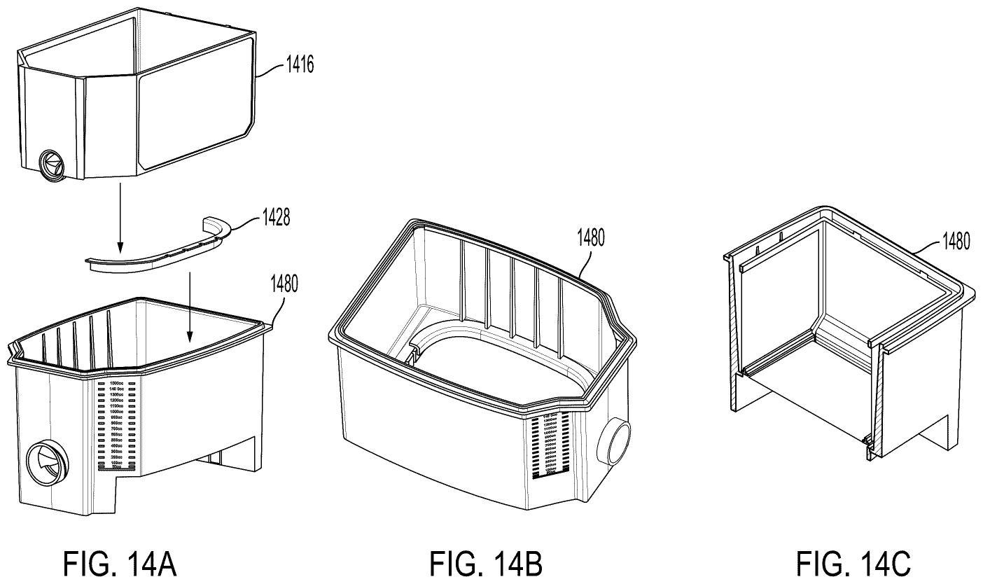

FIGS. 13A-13B illustrates various views of a disclosed system 1300 for washing a lipoaspirate. As shown in FIG. 13, the system 1300 may be graduated along the sides so that a user may be able to determine a volume of contents contained inside the system 1300. FIGS. 14A-14C illustrate various views of a disclosed chamber 1480 without the lid, also presenting the graduated markings. In some embodiments, as shown in FIGS. 14A-14C, the disclosed chamber 1480 may include a reinforced outer wall, shown in FIG. 14C. The reinforced outer wall of the chamber 1480 advantageously prevents cracking when the system is under sustained liposuction pressure.

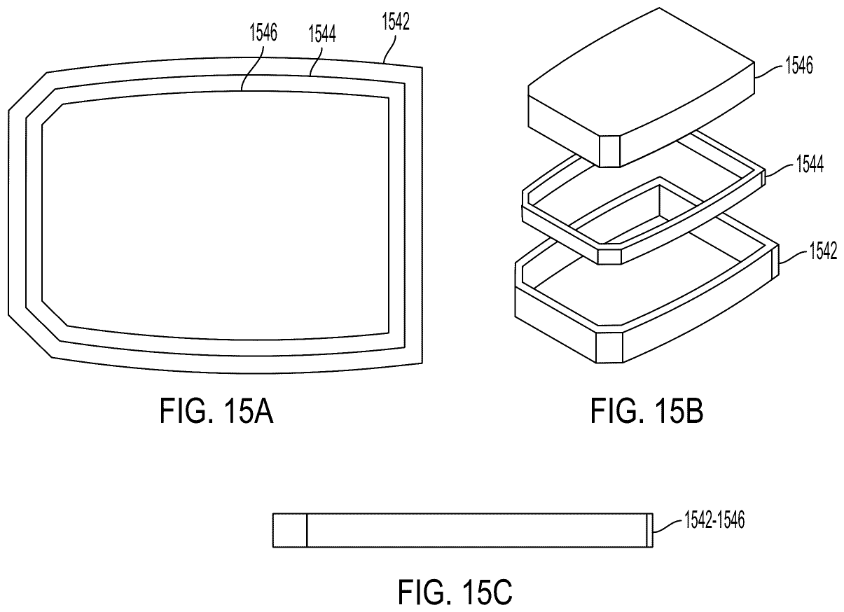

FIGS. 15A-C illustrate various views of a disclosed foam 1546 held into place by container elements 1542 and 1544. The foam may be contained by an outer element 1544 and an inner element 1542.

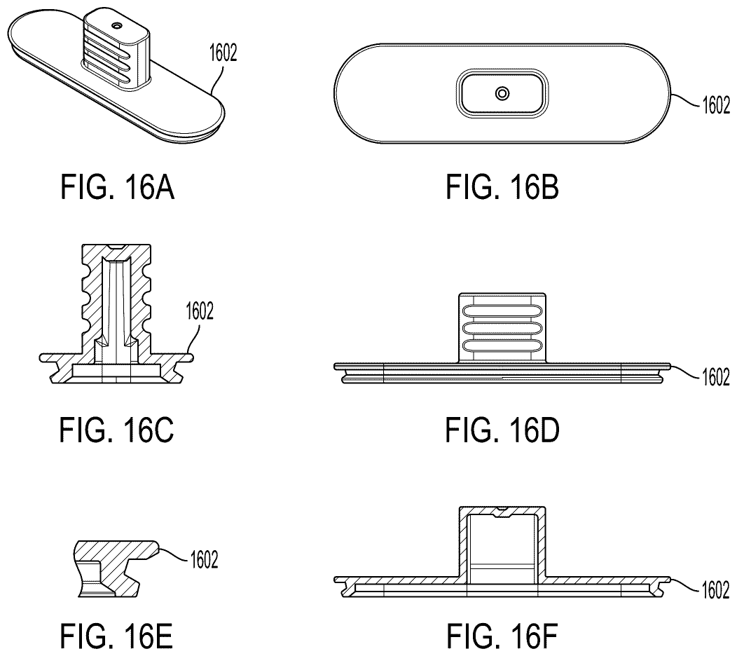

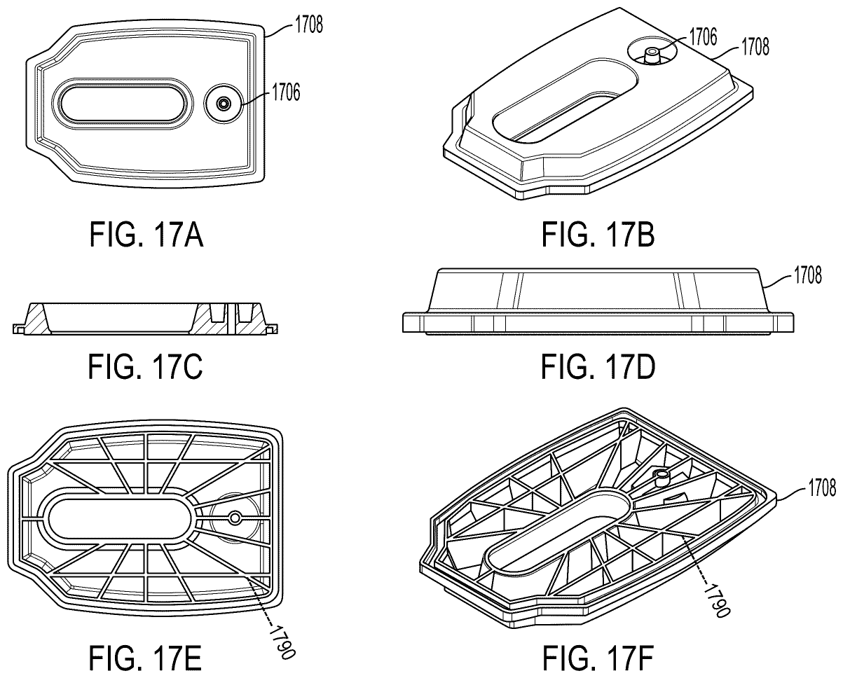

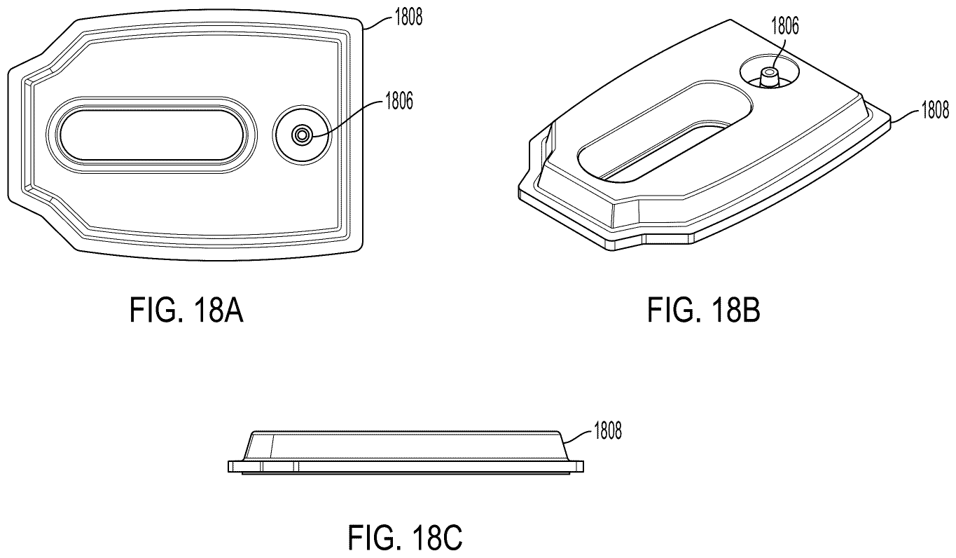

FIGS. 16A-16F illustrate various views of a port cap 1602 for inserting into a lid of a system for washing a lipoaspirate. The port cap 1602 may include a substantially flat base and a handle protruding outward from a top surface of the base. A bottom surface of the base of the port cap 1602 may be configured to fit into a top surface of the lid. In some embodiments, the port cap 1602 may be configured to have a port gasket for enhancing a seal in between the port cap 1602 and the lid. FIGS. 17A-18C illustrate various views of a lid 1708 configured to hold the port cap 1602 shown in FIGS. 16A-16F. FIGS. 17A and 17B show the top portion of the harvest port 1706 of the lid 1708. As shown in FIG. 17E-17F, the lid 1708 may include a plurality of ribs 1790 stemming from the port and extending to an inner surface of one of the side walls of the lid. The ribs 1790 protect the lid against cracking while being placed under pressure. The ribs 1790 may permit the lid to sustain suction pressure.

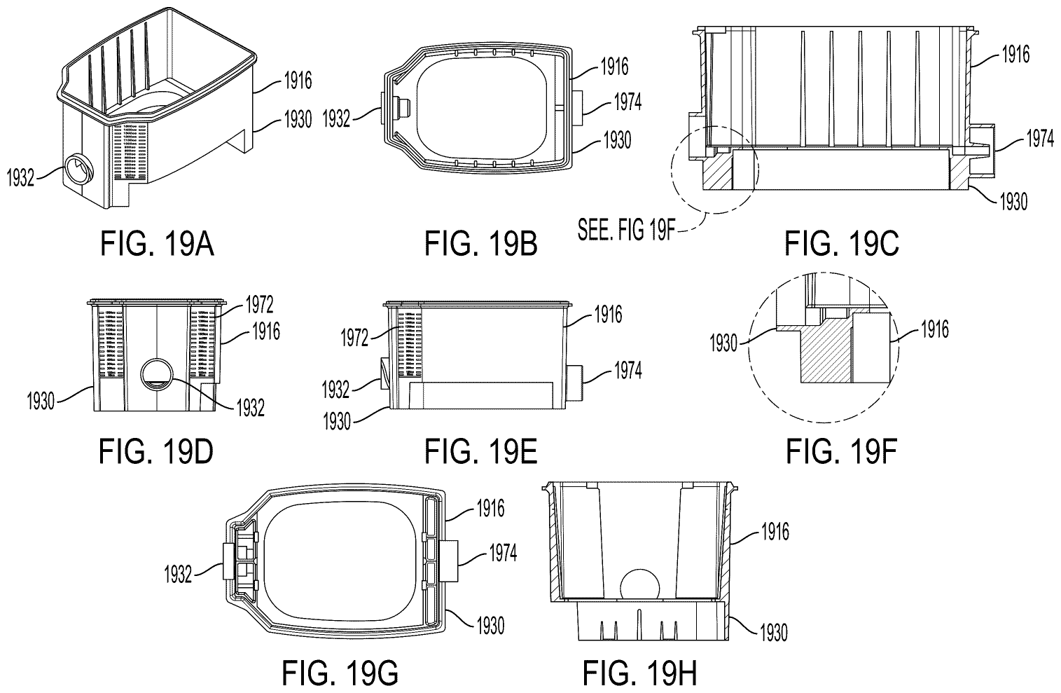

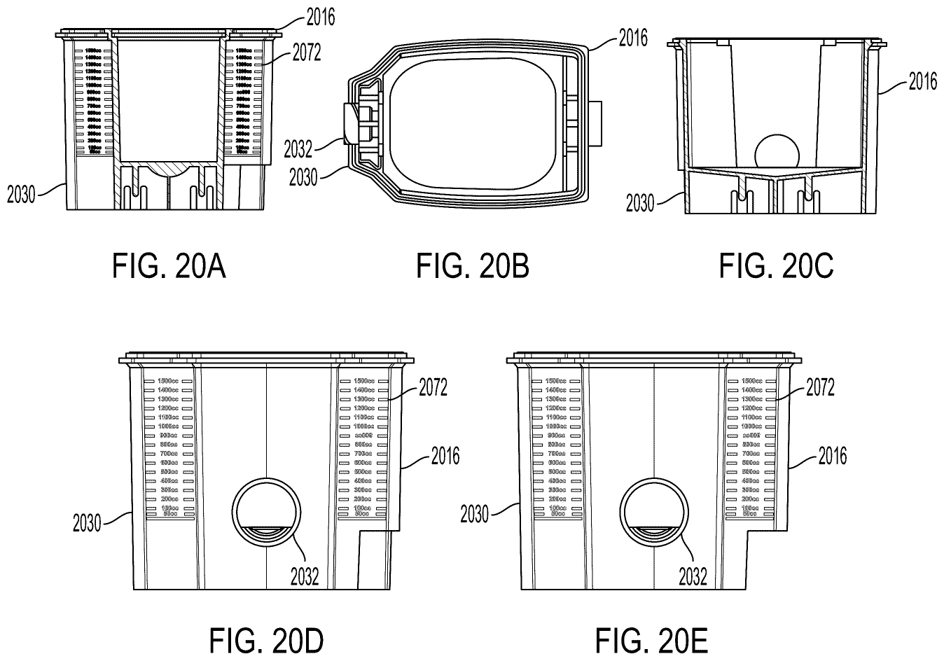

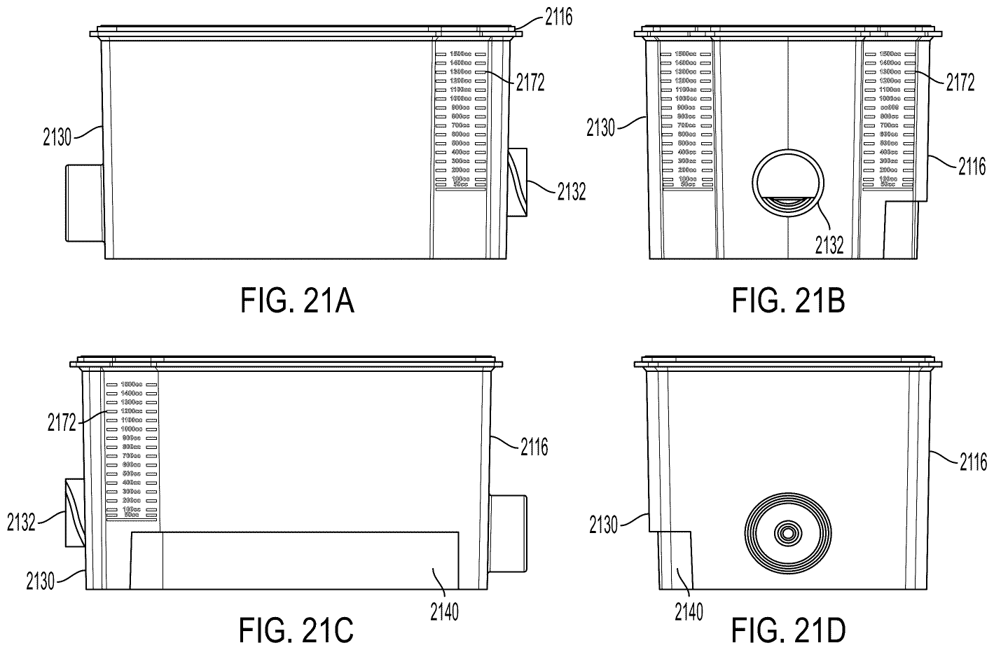

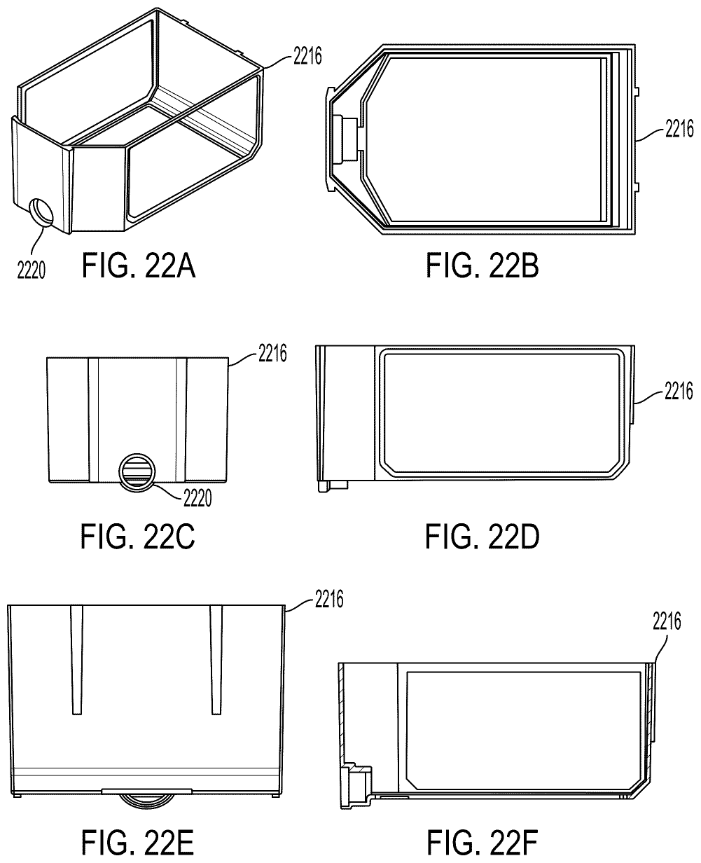

FIGS. 19A-20E illustrate various views of a graduated chamber 1930, 2030. The chamber 1930, 2030 may include graduated markings on one or two sides. In some embodiments, a chamber 1930, 2030 may include threading about the extraction port 1932 for coupling to syringes. The extraction port may include a gasket that provides a seal to prevent pressure loss when providing a suction via syringe. As shown in FIG. 19A-19H, the sub-assembly 1916 may include vertical ridges along an interior surface of the chamber 1916. For example, the sub-assembly 1916 may include from 1 to 10 ridges. FIGS. 19A-20E illustrate a chamber 1930, 2030 not coupled to a removable separator. In contrast, FIGS. 21A-21C illustrate a chamber 2130 coupled to a tray 2140. FIGS. 22A-22F illustrate various views of a sub-assembly 2216.

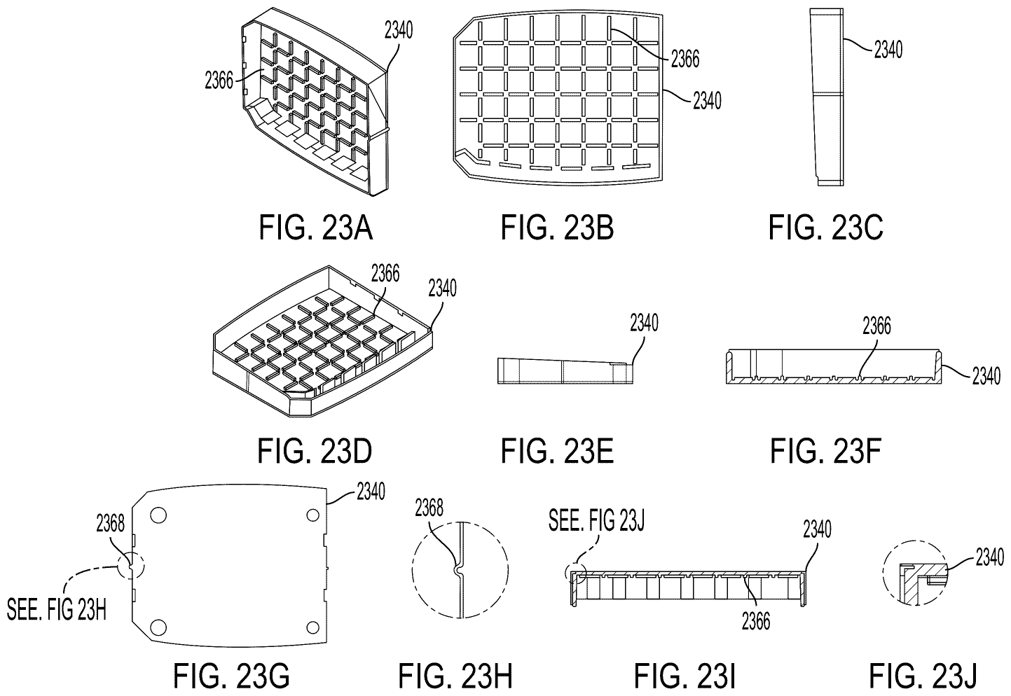

In some embodiments, as shown in FIGS. 23A-23J, a tray 2340 may include channels 2366, which may aid with separating a waste fluid from a washed lipoaspirate. Disclosed channels 2366 may be raised or indented with respect to the tray. In some embodiments, disclosed channels 2366 may be a combination of raised and indented channels, as shown in FIG. 23F. FIGS. 23G-23H show a tray 2340 having a protrusion 2368 along a side of the tray 2340. The protrusion 2368 may facilitate the tray 2340 engaging a chamber when being fit together. The protrusion 2368 may advantageously align parts of the tray 2340 with the chamber when being fitted together.

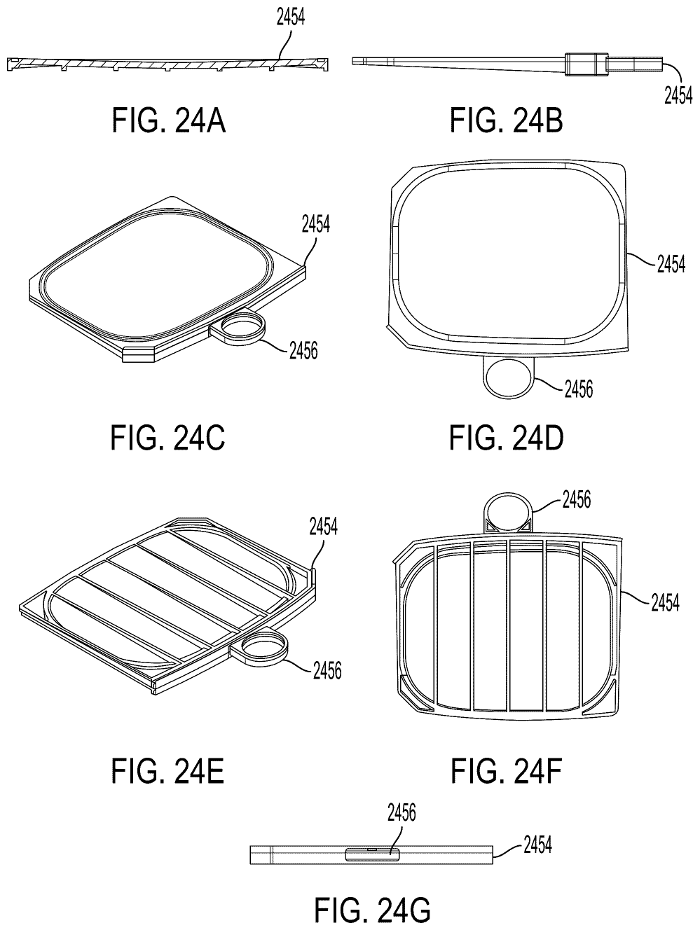

As shown in FIGS. 24A-24G, a disclosed system for washing a lipoaspirate may include a removable separator 2454. The removable separator may include a substantially flat shape, such as being a rectangular prism. The removable separator 2454, as shown in FIGS. 24A, 24E, and 24F may include a series of channels on a bottom side of the removable separator 2454, which may advantageously aid the removable separator 2454 in coupling to contents of a tray when fit together inside of a chamber. As shown in FIGS. 24C-24F, a removable separator 2454 may include a handle 2456, which may protrude outward from a chamber. The handle 2456 may be grabbed by a user to facilitate when pulling the removable separator 2454 out of the chamber. A handle 2456 may be any general shape, such as circular, square, triangle, polygon, cylinder, cube, cuboid, cone, pyramid, prism, and combination thereof. The handle 2456 may be a solid piece, or as shown in FIGS. 24C-24F, may include a hole throughout, such as a circular hole. Having a hole through the handle 2456 may provide easier gripping when positioning the removable separator 2454 into or out of the chamber.

### Method for Washing a Harvested Lipoaspirate

The present disclosure relates to methods for washing a harvested lipoaspirate using disclosed systems, such as those shown in FIGS. 1-24. Disclosed methods involve a combination of washing (e.g., with washing solution, such as an admixture of P188 and saline, or saline and another type of surfactant), mesh filtration, concentration of lipoaspirate, and absorption and removal of waste products from a washing system so that a washed lipoaspirate remains. The washing solution may include an admixture of P188 and saline, or even saline and other surfactants. The washed lipoaspirate may be grafted into a patient.

In some embodiments, the present disclosure relates to a method for washing a harvested lipoaspirate with a lipoaspirate washing system. A method includes receiving the harvested lipoaspirate into a chamber of the lipoaspirate washing system so that the harvested lipoaspirate is contained by a mesh basket disposed in an upper portion of the chamber of the lipoaspirate washing system, thereby forming a received lipoaspirate. The harvested lipoaspirate may be harvested from various donor sites of a patient. The method may include attaching the harvest port to a liposuction cannula.

Once the harvested lipoaspirate has been received into a chamber, a method may include adding a washing solution, such as described in, but not limited to, U.S. Pat. No. 8,512,695, to the chamber and mixing the washing solution with the received lipoaspirate to form a mixed lipoaspirate. Mixing the washing solution with the received lipoaspirate may extract waste products from the viable adipose tissue. The washing solution may include a biocompatible block copolymer, such as a poloxamer surfactant having repeating units of polyethylene oxide (PEO) and polypropylene oxide (PPO). In some embodiments, the copolymer is poloxamer P188. The washing solution may include a saline component, including water combined with any known salt, such as sodium chloride, potassium chloride, and other salts. In some embodiments, mixing the washing solution with the received lipoaspirate may be done with a spatula for a time ranging from about 1 minute to about 10 minutes, or more. For example, mixing may be done for a time of about 1 minute, or about 2 minutes, or about 3 minutes, or about 4 minutes, or about 5 minutes, or about 6 minutes, or about 7 minutes, or about 8 minutes, or about 9 minutes, or about 10 minutes, where about includes plus or minus 0.5 minutes.

According to some embodiments, adding the washing solution to a chamber may be done in various concentrations with respect to the lipoaspirate to form a mixed lipoaspirate. The washing solution may be added to a chamber so that a ratio of the washing solution to the received lipoaspirate ranges from about 1:4 to about 4:1. For example, the ratio of washing solution to received lipoaspirate may be about 1:4, or about 1:3, or about 1:2, or about 1:1, or about 2:1, or about 3:1, or about 4:1, where about includes plus or minus 0.5:0.5.

In some embodiments, adding a washing solution to a chamber containing a harvested lipoaspirate may include pouring the washing solution through an opening in a lid that is securely connected to a top side of a chamber of the lipoaspirate washing system. Adding a washing solution may include pouring the washing solution directly into a chamber where no lid is present. The lid may also include a port cap that may be pulled away from the lid, providing the opening. Replacing the port cap may seal off the lid. In addition to pouring, the washing solution may be injected or pumped using any known means of fluid transport.

After mixing a washing solution with a received lipoaspirate to form a mixed lipoaspirate, a method may include separating a mixed lipoaspirate into an extracted waste fluid and a washed lipoaspirate. The extracted waste fluid may include blood, oil, water, and other unwanted waste products that may reduce the success of the washed lipoaspirate being used for fat grafting.

In some embodiments, washed lipoaspirate and/or concentrated lipoaspirate may be removed from a lipoaspirate washing system from an extraction port disposed on a lower portion of a side wall of a chamber. The concentrated lipoaspirate may have a higher concentration of lipoaspirate per volume in comparison to a harvested lipoaspirate. The concentrated lipoaspirate may have a concentration of lipoaspirate per unit volume that is from about 2 times to about 100 times higher than the harvested lipoaspirate. The concentrated lipoaspirate may have a concentration of lipoaspirate per unit volume that is 2 times higher, or about 10 times higher, or about 20 times higher, or about 30 times higher, or about 40 times higher, or about 50 times higher, or about 60 times higher, or about 70 times higher, or about 80 times higher, or about 90 times higher, or about 100 times higher, than the harvested lipoaspirate, where about includes plus or minus 5 times.

According to some embodiments, a removable separator may separate a mixed lipoaspirate contained in a mesh basket located in a top portion of a chamber from a tray and extraction port both located in a bottom portion of the chamber. A method may include removing the removable separator from the lipoaspirate washing system so that washed aspirate may contact the foam. A user may pull a handle protruding from a side of a removable separator, thereby removing the removable separator from a side of the system. A portion of the extracted waste product may be absorbed into a foam assembly contained within the tray to form an absorbed waste that may be separately discarded from a lipoaspirate washing system. Absorbing the extracted waste product into the foam assembly may concentrate the washed aspirate, forming a concentrated lipoaspirate. The concentrated lipoaspirate may then be removed from the washing system from the extraction port disposed on the lower portion of the side wall of the chamber.

According to some embodiments, disclosed methods of washing a lipoaspirate may be iterative in that they may be performed multiple times in batch or in flow. For example, a method may be run once so that a washed lipoaspirate is formed and extracted waste fluid is removed from a disclosed system, then additional harvested lipoaspirate may be added to the chamber for washing. This may be used to increase a yield of washed lipoaspirate. Disclosed methods may include steps of receiving additional harvested lipoaspirate into the chamber of the lipoaspirate washing system so that the additional harvested lipoaspirate is contained by the mesh basket disposed in the upper portion of the chamber of the lipoaspirate washing system to form additional received lipoaspirate. In batch, disclosed systems and methods may be able to wash from about 10 mL of harvested lipoaspirate to about 2,000 mL of harvested lipoaspirate. For example, disclosed systems and methods may wash about 10 mL, or about 200 mL, or about 400 mL, or about 600 mL, or about 800 mL, or about 1,000 mL, or about 1,200 mL, or about 1,400 mL, or about 1,600 mL, or about 1,800 mL, or about 2,000 mL of harvested lipoaspirate, where about includes plus or minus 100 mL. In an embodiment, a disclosed system or method may wash about 1,050 mL of a harvested lipoaspirate. Alternatively, washed lipoaspirate may be continuously removed from the disclosed system as new batches of harvested lipoaspirate is added so the system is run in flow instead of batchwise.

Disclosed methods may include a step of adding additional washing solution(s) to the chamber and mixing the washing solution with the lipoaspirate with a spatula or other mixing device to form additional amounts of mixed lipoaspirate. In some embodiments, disclosed methods may include separating additional waste fluid from the additional mixed lipoaspirate to form additional amounts of extracted waste fluid and additional amounts of washed lipoaspirate, wherein the extracted waste fluid is removed from the lipoaspirate washing system from an extraction port disposed on a lower portion of a side wall of the chamber.

The terminology used in the description herein is for the purpose of describing particular embodiments and is not intended to be limiting. As used in the description of the invention and the appended claims, the singular forms “a,” “an” and “the” are intended to include the plural forms as well, unless the context clearly indicates otherwise. It will also be understood that the term “and/or” as used herein refers to and encompasses any possible combination of one or more of the associated listed items. It will be further understood that the terms “includes,” “including,” “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “if” may be construed to mean “when” or “upon” or “in response to determining” or “in response to detecting,” depending on the context.

Those with skill in the art will appreciate that while some terms in this disclosure may refer to absolutes, the methods and techniques disclosed herein may also be performed on fewer than all of a given thing, e.g., performed on one or more components. Accordingly, in instances in the disclosure where an absolute is used, the disclosure may also be interpreted to be referring to a subset.

What is claimed is:

1. A system for washing lipoaspirate, comprising:
(a) a chamber comprising a hollow body formed of side walls having an exterior surface and an interior surface, the chamber being open at a top side and a bottom side of the chamber;
(b) a lid comprising:
(i) a top surface;
(ii) a lip extending downward from a bottom surface of the lid and configured to securely connect the lid to the top side of the chamber, and
(iii) a harvest port comprising a cylindrical tap extending outwardly from the top side of the lid and configured to connect to and receive a harvested lipoaspirate from a hose;
(c) a sub-assembly comprising a mesh basket and disposed in an upper portion of the chamber, the sub-assembly configured to receive the lipoaspirate received through the harvest port and to separate extracted waste fluid from the lipoaspirate to form a washed lipoaspirate;
(d) a tray configured to insert into a lower portion of the chamber through an opening in the side walls, thereby forming a bottom portion of the system, the tray comprising:
(i) a plate;
(ii) raised sidewalls positioned along the entire perimeter of the plate and extending upward from a top surface of the plate; and
(ii) a foam assembly fitting onto the plate and within the sidewalls so that the foam assembly is disposed in a lower portion of the chamber and configured to concentrate the washed lipoaspirate; and
(e) an extraction port along one of the side walls of the chamber configured to permit removal of lipoaspirate that has been one of washed and concentrated.

2. The system for washing lipoaspirate according to claim 1,
wherein at least one of:
the extraction port comprises a cylindrical hole in the lower portion of the chamber that protrudes outward from the side of the chamber, and
the extraction port contains threads configured to couple to an extraction port cap that contains the extraction within the system.

3. The system for washing lipoaspirate according to claim 1, wherein the lid further comprises a partition for storing at least one of a spatula and a cleaning packet.

4. The system for washing lipoaspirate according to claim 1,
wherein the system further comprises a removable separator positioned on a top surface of the raised sidewalls of the tray, the removable separator configured to separate contents contained within in the chamber from the tray, and
wherein removable separator comprises:
a gasket configured to form a seal between the removable separator and the chamber; and
a handle protruding outward from a side of the removable separator and thus outward from the chamber, wherein the handle is configured to be pulled to remove the removable separator from the chamber.

5. The system for washing a lipoaspirate according to claim 1,
wherein the foam assembly comprises a plurality of foam members, forming a complex channel system configured to redistribute a fluid from the lipoaspirate throughout the foam assembly, and
wherein the foam assembly is configured to prevent saturation of the foam members before they are able to concentrate the lipoaspirate.

6. The system for washing a lipoaspirate according to claim 1, wherein the lid comprises an opening on a top surface of the lid configured to permit access to an inside of the mesh basket.

7. The system for washing a lipoaspirate according to claim 1, wherein the mesh basket comprises a mesh bottom forming a bottom portion of the sub-assembly and a plurality of mesh sides forming side portions of the sub-assembly.

8. The system for washing a lipoaspirate according to claim 1, wherein the tray further comprises a squeegee.

9. The system for washing a lipoaspirate according to claim 1, wherein the lid further comprises a port cap fitting into an opening on a top surface of the lid and configured to be removed from the opening permit access to an inside of the chamber by the user with a spatula.

10. The system for washing a lipoaspirate according to claim 1,
wherein the sub-assembly comprises a circular hole in a lower portion of the sub-assembly that lines up with extraction port of the chamber, and
wherein the circular hole is configured to form a seal with the extraction port through a slit valve.

11. A method for washing a harvested lipoaspirate with a lipoaspirate washing system, the method comprising:
(a) receiving the harvested lipoaspirate into a chamber of the lipoaspirate washing system so that the harvested lipoaspirate is contained by a mesh basket disposed in an upper portion of the chamber of the lipoaspirate washing system to form a received lipoaspirate;
(b) adding a washing solution to the chamber and mixing the washing solution with the received lipoaspirate using a spatula to form a mixed lipoaspirate; and
(c) separating the mixed lipoaspirate into an extracted waste fluid and a washed lipoaspirate.

12. The method for washing the lipoaspirate according to claim 11, further comprising:
(d) removing a removable separator from the lipoaspirate washing system so that washed aspirate may lower onto a foam assembly contained in a tray contained in a lower portion of the chamber and located below the mesh basket; and
(e) absorbing a portion of the extracted waste fluid into the foam assembly to concentrate the washed aspirate, forming a concentrated lipoaspirate,
wherein one of the washed lipoaspirate and the concentrated aspirate is removed from the lipoaspirate washing system from an extraction port disposed on a lower portion of a side wall of the chamber.

13. The method for washing the lipoaspirate according to claim 11,
wherein the concentrated lipoaspirate comprises a concentration of lipoaspirate per unit volume that is from about 2 to about 100 times higher than the harvested lipoaspirate.

14. The method for washing the lipoaspirate according to claim 11, further comprising attaching the harvest port to a liposuction cannula.

15. The method for washing the lipoaspirate according to claim 11,
wherein the adding the washing solution to the chamber comprises pouring the washing solution through an opening in a lid that securely connects to a top side of the chamber through a lip extending downward from a bottom surface of the lid.

16. The method for washing the lipoaspirate according to claim 11, wherein at least one of:
the adding a washing solution to the chamber is done in a ratio of the washing solution to the received lipoaspirate that ranges from about 1:4 to about 4:1, and
the mixed lipoaspirate sits for a time ranging from about 30 seconds to about five minutes.

17. The method for washing the lipoaspirate according to claim 11, wherein one of:
the mixing the washing solution with the received lipoaspirate with a spatula occurs for a time ranging from about 1 minutes to about 10 minutes, and
the washing solution comprises a biocompatible block copolymer comprising repeating units of Polyethylene oxide (PEO) and polypropylene oxide (PPO).

18. The method for washing the lipoaspirate according to claim 11, further comprising:
(d) receiving additional harvested lipoaspirate into the chamber of the lipoaspirate washing system so that the additional harvested lipoaspirate is contained by the mesh basket disposed in the upper portion of the chamber of the lipoaspirate washing system to form additional received lipoaspirate.

19. The method for washing the lipoaspirate according to claim 18, further comprising:
(e) adding additional washing solution to the chamber and mixing the washing solution with the received lipoaspirate with the spatula to form additional mixed lipoaspirate.

20. The method for washing the lipoaspirate according to claim 19, further comprising:
(f) separating additional waste fluid from the additional mixed lipoaspirate to form additional extracted waste fluid and additional washed lipoaspirate, wherein the extracted waste fluid is removed from the lipoaspirate washing system from an extraction port disposed on a lower portion of a side wall of the chamber.
